(19) United States
(12) Patent Application Publication
(10) Pub. No.: US 2026/0344860 A1
(43) Pub. Date: Nov. 5, 2026

(54) METHOD OF DYEING HAIR THROUGH PHYSICAL ADSORPTION OF PIGMENT PARTICLES BY CHANGING SURFACE POTENTIAL OF HAIR

(71) Applicant: Gwangju Institute of Science and Technology, Gwangju (KR)

(72) Inventors: Hyeon-Ho JEONG, Gwangju (KR); Doeun KIM, Tongyeong-si (KR); Ji Yeong MA, Jeollanam-do (KR)

(21) Appl. No.: 19/562,919

(22) Filed: Mar. 11, 2026

(30) Foreign Application Priority Data
Apr. 30, 2025 (KR) 10-2025-0057188

(51) Int. Cl. A45D 19/00; A61Q 5/10

(52) U.S. Cl. CPC A45D 19/0066; A61Q 5/10

(57) ABSTRACT

Embodiments relate to a method of dyeing hair through physical adsorption of pigment particles by changing the surface potential of the hair. According to one embodiment, it is possible to dye hair in a simple and quick manner by completely immersing the hair in a dyeing solution and maintaining the immersion state for 60 seconds or less, not only dye the hair in various colors but also achieve a high color coverage, eliminate the use of oxidizing agents (e.g., hydrogen peroxide) and oxidative dye precursors used in conventional oxidative hair dyeing, and enhance user convenience.

### CROSS REFERENCE TO RELATED APPLICATION

The present application claims priority to Korean Patent Application No. 10-2025-0057188, filed Apr. 30, 2025, the entire contents of which are incorporated here for all purposes by this reference.

### BACKGROUND

### 1. Technical Field

The present disclosure relates to a method of dyeing hair through physical adsorption of pigment particles by changing the surface potential of the hair.

### 2. Related Art

Gray hair is not favorable in aesthetic terms and, in some countries, is perceived as a sign of aging, and thus can lead to psychological stress, such as low self-esteem. Although the exact etiology of gray hair still remains unclear, recent research suggests that hydrogen peroxide (H₂O₂) accumulated in the scalp hair shaft damages hair follicles, causing hair color to change. Known factors that cause hair follicle damage include ultraviolet rays, radiation, psychological stress, alcohol consumption, smoking, and inflammation.

Today's hair dyes can be classified into oxidative hair dyes, which are permanent hair dyes, non-oxidative hair dyes, which are semi-permanent hair dyes, and temporary hair dyes, depending on the durability of the dyed color and the characteristics of the composition.

Oxidative hair dyes are based on the principle that low-molecular-weight oxidative dyes or pigments form coloring matter by undergoing an oxidative polymerization reaction within the hair, and are most widely used because they can express a variety of colors and have excellent dyeing power. These oxidative hair dyes contain at least one dye or pigment precursor that forms a dye or pigment by being oxidized by an oxidizing agent, and at least one coupler. These oxidative hair dye compositions are mixed with an oxidizing agent consisting of a hydrogen peroxide solution or the like immediately before application to hair. The purpose of using these oxidation-type hair dyes is to completely dye hair and maintain the dyeing effect for a long period of time.

The oxidative hair dyes currently on the market exhibit excellent color durability because the oxidative dyes that have penetrated the hair undergo a polymerization reaction and become larger in molecular size, making them unable to escape the hair cuticle. However, they easily discolor due to fading caused by repeated shampooing, and alkaline agents and hydrogen peroxide can have a negative effect in terms of hair damage. Furthermore, the oxidative dyes can cause irritation, such as hypersensitivity of the scalp, in addition to hair damage. If the dye gets into the eyes, it is likely to cause serious side effects on the eyes.

In addition, non-oxidative hair dyes cause relatively less hair damage since they do not use oxidizing agents such as hydrogen peroxide, and can be conveniently used. Due to these advantages, the use thereof is expanding. However, the non-oxidative hair dyes still require improvement due to problems such as low dyeing properties and low color durability.

To overcome these problems, products that can exhibit a dyeing effect while being used as hair cleansers rather than as separate dyes, such as Modamoda shampoo, have become increasingly popular in the market.

However, the Ministry of Food and Drug Safety, which reviewed the chemical safety of 1,2,4-trihydroxybenzen (THB) in relation to genotoxicity, notified it as a prohibited ingredient for use in cosmetics, and thus controversy over the hazards of dyeing shampoos has arisen.

Therefore, there is a need to develop a dyeing method that completely excludes the use of chemicals as described above.

### PRIOR ART DOCUMENTS

### Patent Documents

(Patent Document 1) KR 10-2023-0008628 A1

### SUMMARY

An object of the present disclosure is to provide a method of dyeing hair through physical adsorption of pigment particles by changing the surface potential of the hair.

Another object of the present disclosure is to provide a method of dyeing hair, which is capable of dyeing hair by completely immersing the hair in a dyeing solution and maintaining the immersion state for 60 seconds or less, and which not only can dye the hair in various colors but also achieve a high color coverage, and which does not use require oxidative dye precursors and/or an oxidizing developer (e.g., hydrogen peroxide) for dyeing and enhances user convenience.

Still another object of the present disclosure is to provide a method of dyeing hair, which can increase color durability by immersing hair in a dyeing solution, and then immersing the hair in a pigment particle fixing solution for fixing pigment particles without primary washing with water or a separate process, causing a particle fixing layer to be physically adsorbed on pigment particles adsorbed on the hair.

To achieve the above-described objects, the present disclosure relates to a method of dyeing hair, including: a step of dyeing hair by immersing the hair in a dyeing solution containing an acidity regulator and pigment particles, causing the pigment particles to be physically adsorbed on the hair; and a step of immersing the dyed hair in a pigment particle fixing solution, wherein the step of dyeing the hair changes the surface potential of the hair by the dyeing solution, causing the pigment particles to be physically adsorbed on the hair.

In addition, the step of immersing the dyed hair in the pigment particle fixing solution may form a particle fixing layer between the pigment particles, adsorbed on the hair, by the pigment particle fixing solution.

In addition, the dyeing solution may further contain citrate ions.

In addition, the dyeing solution may contain 0.01 mM to 20 mM of the acidity regulator, 0.01 mM to 100 mM of citrate ions, and 0.1 wt % to 0.2 wt % of the pigment particles.

In addition, the pigment particles may be selected from the group consisting of Fe₃O₄, α-Fe₂O₃, FeOOH, Cr₂O₃, Cr₂O₃·xH₂O, lake pigment, and mixtures thereof.

In addition, the pigment particles may have a diameter of 500 μm or less.

In addition, the acidity regulator may be selected from the group consisting of sodium bisulfite, succinic acid, aminomethyl propanediol, acetic acid, disodium phosphate, triethanolamine, and mixtures thereof.

In addition, the pigment particle fixing solution may be selected from the group consisting of polyvinylpyrrolidone (PVP), polyvinyl alcohol (PVA), a cellulose derivative, chitosan, alginate, and mixtures thereof.

In addition, the concentration of the pigment particle fixing solution may be 20 wt % or less.

In addition, the method may further include, after the step of dyeing the hair, but before the step of immersing the dyed hair in the pigment particle fixing solution, a step of washing the dyed hair.

In addition, the method may further include, after the step of immersing the dyed hair in the pigment particle fixing solution, a step of washing the hair.

In addition, the step of dyeing hair by immersing hair in the dyeing solution containing the acidity regulator and the pigment particles, causing the pigment particles to be physically adsorbed on the hair, may dye the hair by immersing the hair in the dyeing solution for 60 seconds or less.

To achieve the above-described objects, the present disclosure relates to a hair dyeing composition including: a dyeing solution containing an acidity regulator and pigment particles; and a pigment particle fixing solution, wherein the surface potential of the hair is changed by the dyeing solution, causing the pigment particles to be physically adsorbed on the hair, so that the hair may be dyed, and the color durability of the dyed hair is increased by immersing the dyed hair in the pigment particle fixing solution.

In addition, the dyeing solution may further contain citrate ions.

According to the present disclosure, it is possible to dye hair in a simple and quick manner by completely immersing the hair in a dyeing solution and maintaining the immersion state for 60 seconds or less, not only dye the hair in various colors but also achieve a high color coverage, eliminate the use of an oxidizing agent (e.g., hydrogen peroxide) and oxidative dye precursors commonly used in conventional oxidative hair dyeing, and enhance user convenience.

In addition, it is possible to increase color durability by immersing hair in a dyeing solution, and then immersing the hair in a pigment particle fixing solution for fixing pigment particles without primary washing with water or a separate process, causing a particle fixing layer to be physically adsorbed on pigment particles adsorbed on the hair.

### BRIEF DESCRIPTION OF THE DRAWINGS

FIG. 1 shows a method of dyeing hair according to one embodiment of the present disclosure.

FIG. 2 shows the results of an experiment on the degree of dyeing depending on dyeing time for artificial hair according to one example of the present disclosure.

FIG. 3 shows the results of an experiment on the degree of dyeing depending on dyeing time for human hair according to one example of the present disclosure.

FIG. 4 shows the results of measuring reflectance depending on dyeing time for artificial hair according to one example of the present disclosure.

FIG. 5 shows the results of measuring reflectance depending on dyeing time for human hair according to one example of the present disclosure.

FIG. 6 shows the results of dyeing depending on the concentration change of an acidity regulator and on dyeing time according to one example of the present disclosure.

FIG. 7 shows the results of measuring the change in surface potential depending on the proton concentration of hair and pigment particles according to one example of the present disclosure.

FIG. 8 shows the generation of a potential difference confirmed by measuring the surface potential of pigment particles and hair according to one example of the present disclosure.

FIG. 9 shows the results of an experiment on adherence rate depending on a change in the concentration of an acidity regulator and a difference in coating time according to one example of the present disclosure.

FIG. 10 shows a dyeing color for artificial hair depending on the type of pigment particles according to one example of the present disclosure.

FIG. 11 shows a dyeing color for human hair depending on the type of pigment particles according to one example of the present disclosure.

FIG. 12 is an SEM photograph of hair dyed using a dyeing solution according to one example of the present disclosure.

FIG. 13 is an SEM photograph of hair after dyeing using a dyeing solution and then forming a particle fixing layer using a pigment particle fixing solution, according to one example of the present disclosure.

FIG. 14 shows the results of measuring the reflectance of hair after dyeing using a dyeing solution and then forming a particle fixing layer using a pigment particle fixing solution, according to one example of the present disclosure.

FIGS. 15 and 16 show the results of a hair dyeing durability test according to the presence or absence of a particle fixing layer using a pigment particle fixing solution after dyeing using a dyeing solution according to one embodiment of the present invention.

### DETAILED DESCRIPTION

Hereinafter, embodiments of the present disclosure will be described in detail so that those skilled in the art can easily implement the present disclosure. However, the present disclosure may be embodied in various different forms and is not limited to the embodiments described herein.

The current hair care and dyeing industry and related markets are growing rapidly across all age groups, from young to old, due to the growing aging population and trends, such as hair fashion trends among young consumers, and increasing demand for natural and chemical-free hair coloring products.

In particular, interest in the hair dye and hair care market has been rapidly increasing, with the domestic launch of Modamoda hair dye shampoo and its domestic and international sales reaching 60 billion won, and investment and competition among beauty companies such as Amorepacific and LG Household & Health Care to catch up are intensifying.

However, as the Ministry of Food and Drug Safety, which reviewed the chemical safety of 1,2,4-trihydroxybenzen (THB) in relation to genotoxicity, notified it as a prohibited ingredient for use in cosmetics, and furthermore, as the Ministry of Food and Drug Safety announced an administrative notice of a revision to the “Regulations on Cosmetic Safety Standards, etc.” that designates five ingredients including o-aminophenol, m-phenylenediamine hydrochloride, m-phenylenediamine, catechol, and pyrogallol as ingredients that cannot be used in cosmetics, controversy over the hazards of late dyeing shampoos has also arisen.

In particular, products for seniors target the elderly with weak immune systems, so they require ingredients that are friendly to the human body by containing a reduced amount of chemicals.

Therefore, the present disclosure is intended to propose a hair dyeing technique that avoids oxidative dyeing chemistry, for example, without requiring oxidative dye precursors and an oxidizing developer, and instead achieves coloring by electrostatic assembly of pigment particles.

Accordingly, the present disclosure is intended to provide a method of dyeing hair, including: a step of dyeing hair by immersing the hair in a dyeing solution containing an acidity regulator and pigment particles, causing the pigment particles to be physically adsorbed on the hair; and a step of immersing the dyed hair in a pigment particle fixing solution, wherein the step of dyeing the hair changes the surface potential of the hair by the dyeing solution, causing the pigment particles to be physically adsorbed on the hair.

As described above, according to the present disclosure, hair dyeing is completed by simply immersing the hair in the dyeing solution, indicating that the dyeing method is simpler than conventional hair dyes. In addition, by further immersing the hair in the pigment particle fixing solution after immersing it in the dyeing solution, the adherence of the pigment particles adsorbed on the hair may be further enhanced, thereby increasing color durability, and even if direct impact is applied to the hair, the problem that the adsorbed pigment particles fall off does not occur.

Compared to conventional hair dyes, the present disclosure may ensure user convenience equivalent to that of a hair dye shampoo because the dyeing process is very simple. In the case of hair dye shampoos, the dyeing effect is insufficient, so that users are not completely satisfied with the hair dyeing effect. However, the present disclosure may exhibit a hair dyeing effect equivalent to that of a hair dye, and thus exhibit a color coverage level equivalent to that of conventional hair dyes.

This means that the dyeing method of the present disclosure may utilize only the advantages of the hair dye and the advantages of the hair dyeing shampoo.

The dyeing method of the present disclosure utilizes the property of physically adsorbing the pigment particles on hair by changing the surface potential of the hair by the dyeing solution. Specifically, the dyeing solution contains an acidity regulator and pigment particles, wherein the acidity regulator maintains the dyeing solution in an acidic state. In the acidic state, the dyeing solution contains a large number of proton ions. The proton ions play a role in dissociating the OH— ion groups on the hair surface. When the OH— ion groups on the hair surface are dissociated as described above, the surface potential of the hair is reversed to positive. When the surface potential of the hair is reversed to positive, the electrostatic potential difference between the negatively charged pigment particles and the hair surface whose potential has been switched to a positive value increases, so that the pigment particles are attracted to the hair surface by electrostatic attraction, resulting in physical adsorption.

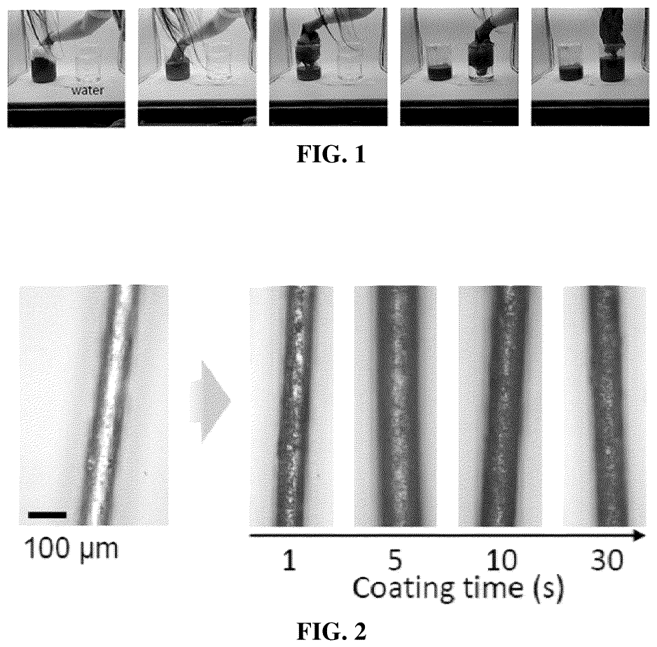

Specifically, as the surface potential of hair changes as shown in FIG. 1, a potential difference with respect to the pigment particles occurs. According to this potential difference, electrostatic attraction occurs, so that physical adsorption of the pigment particles on the hair surface occurs

The physical adsorption as described above causes a reaction to occur at a high adherence rate. Thus, according to the hair dyeing method of the present disclosure, hair may be dyed by immersing the hair in the dyeing solution for 60 seconds or less, 50 seconds or less, 40 seconds or less, 30 seconds or less, 20 seconds or less, or 10 seconds or less.

Since hair dyeing is carried out by physical adsorption on the hair, the pigment particles may have a particle diameter of 500 μm or less, 450 μm or less, 400 μm or less, 350 μm or less, 300 μm or less, 250 μm or less, 200 μm or less, 150 μm or less, 100 μm or less, or several tens of μm or less. By using the pigment particles having a diameter within the above-described range, the hair dyeing effect may be exhibited within a short period of time.

The dyeing solution may further contain citrate ions. The citrate ions act not only as a surfactant that helps maintain the dispersion stability of the dye particles in the dyeing solution, but also as buffer ions. A buffer solution containing buffer ions refers to a solution in which the hydrogen ion concentration (pH) of the solution does not change significantly due to the common ion effect even when an acid or base is added thereto. The applicable pH range varies depending on the type of buffer ion, and citrate acts as a buffer in the range of pH 2.2 to pH 6.5. The citrate ions may form sodium citrate in the dyeing solution.

The dyeing solution may contain 0.01 mM to 20 mM of the acidity regulator, 0.01 mM to 100 mM of citrate ions, and 0.1 wt % to 0.2 wt % of the pigment particles. The acidity regulator is intended to change the surface potential of the hair as described above. If the content of the acidity regulator is higher than the upper limit of the above range, the acidity regulator may be harmful to the human body because it may be a strong acid with a low pH. If the content of the acidity regulator is lower than the lower limit of the above range, positive charging of the hair surface may not easily occur, and thus the effect of adsorbing the pigment particles may be insufficient. If the citrate ions are contained in an amount larger than the upper limit of the above range, the stability of the pigment particles may be lowered, causing agglomeration, due to the high ionic strength of the citrate ions, and if the citrate ions are contained in an amount smaller than the lower limit of the above range, the stability of the pigment particles in the dyeing solution may be lowered. In addition, if the pigment particles are contained in an amount larger than the upper limit of the above range, a problem may arise in that the viscosity of the dyeing solution increases, so that the pigment particles do not exist in a dispersed state and the effect due to viscosity is enhanced, and thus when the hair is immersed in the dyeing solution and dried, the hair and the pigment particles excessively clump. If the pigment particles are contained in an amount smaller than the lower limit of the above range, a problem may arise in that a lot of time is required for complete hair dyeing.

In addition, the pigment particles may be selected from the group consisting of Fe₃O₄, α-Fe₂O₃, FeOOH, Cr₂O₃, Cr₂O₃·xH₂O, lake pigment, and mixtures thereof. However, the pigment particles are not limited to the above examples, and may include any pigment powder permitted for cosmetic use.

The pigment particles are optionally contained in the dyeing solution, and there is a difference in color depending on the pigment particles. Fe₃O₄ can show black, α-Fe₂O₃ can shows red, FeOOH and lake pigment can show yellow, and Cr₂O₃ and Cr₂O₃·xH₂O can show blue. These colors may be implemented in various colors by combining pigment particles. In addition, in addition to the pigment particles exemplified above, any pigment particles that may achieve hair coloring by physical adsorption on positively charged hair may be used without limitation.

The acidity regulator may be selected from the group consisting of sodium bisulfite, succinic acid, aminomethyl propanediol, acetic acid, disodium phosphate, triethanolamine, and mixtures thereof, and is preferably sodium bisulfite. However, the acidity regulator is not limited to the above examples, and any acidity regulator that may be used in foods or cosmetics may be used without limitation.

As described above, after completion of the step of dyeing the hair by immersing the hair in the dyeing solution, a subsequent step of immersing the dyed hair in a pigment particle fixing solution may be performed. However, prior to performing the step of immersing the dyed hair, a primary washing step may be additionally performed to remove any pigment particles and dyeing solution remaining after the step of dyeing the hair. However, the washing step is optional.

After completion of the step of dyeing the hair, in the present disclosure, the step of immersing the dyed hair in the pigment particle fixing solution as described above may be performed.

The step of immersing the hair in the pigment particle fixing solution is to form a particle fixing layer between the pigment particles adsorbed on the hair by immersing the pigment particle-adsorbed hair in the pigment particle fixing solution.

The hair dyeing process of the present disclosure is not a chemical hair dyeing process, but is a process that physically adsorbs the pigment particles on the hair to produce color. Therefore, even if the hair dyeing process proceeds sufficiently, a problem may arise in that the pigment particles may fall off if external impact is applied to the hair.

That is, the pigment particles are in a state adsorbed on the hair by electrostatic attraction, and a problem may arise that, if an external impact stronger than the electrostatic attraction is applied, the physical adsorption state is broken. To prevent this problem and further enhance the hair color retention effect, a particle fixing layer may be formed.

The pigment particle fixing solution may be selected from the group consisting of polyvinylpyrrolidone (PVP), polyvinyl alcohol (PVA), a cellulose derivative, chitosan, alginate, and mixtures thereof. However, the pigment particle fixing solution is not limited to the above-described examples, and other bio-stable adhesive materials for cosmetics may also be included.

More specifically, the pigment particle fixing solution may be a polyvinylpyrrolidone (PVP) solution. The polyvinylpyrrolidone concentration of the polyvinylpyrrolidone solution may be 20 wt % or less, 0.01 wt % to 20 wt % or less, 0.1 wt % to 20 wt % or less, or 1 wt % to 20 wt % or less. If the polyvinylpyrrolidone solution has a concentration exceeding the upper limit of the above range, the solution may not be uniformly distributed over the entire hair due to its high viscosity even if the hair is entirely immersed in the polyvinylpyrrolidone solution, making it difficult to form a particle fixing layer.

The method may further include, after the step of immersing the dyed hair in the pigment particle fixing solution, a step of washing the dyed hair.

The above-described method of dyeing hair is advantageous in that it dyes hair by physical adsorption without relying on oxidative dyeing reactions (e.g., oxidative polymerization within hair) and without requiring an oxidizing developer (e.g., hydrogen peroxide), it does not use these chemicals that are harmful to the user. In addition, the method may dye hair quickly, allowing people who need to dye their hair to dye their hair easily.

### Preparation Example

A dyeing solution was prepared by dissolving 1 mM of sodium bisulfite, 0.25 mM of citrate, and 0.15 wt % of Fe₃O₄ in water. A solution containing 5 wt % of polyvinylpyrrolidone (PVP) was used as a pigment particle fixing solution.

### Test Example

### Determination of Whether Hair is Dyed

To determine whether hair is dyed, a hair dyeing process for artificial hair made of PET material and human hair was performed using the dyeing solution and pigment particle fixing solution prepared in the Preparation Example while changing the dyeing time from 1 second to 5 seconds, 10 seconds, and 30 seconds.

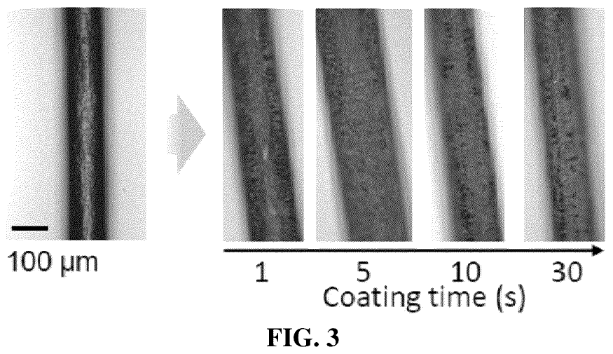

In the hair dyeing process, as shown in FIG. 1, hair was immersed in the dyeing solution, maintained for the dyeing time described above, taken out, washed with water, immersed in the PVP solution, washed, and then completely dried. FIG. 2 only shows the process of dyeing with the dyeing solution and washing.

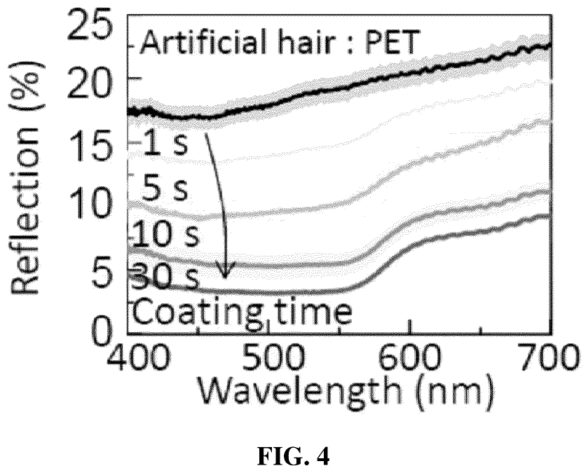

The hair dyeing results are shown in FIGS. 2 and 3. Specifically, FIG. 2 shows the dyeing results for artificial hair, and FIG. 3 shows the dyeing results for human hair. According to the test results, it can be confirmed that the pigment particles were adsorbed on almost the entire surface of the hair after 5 seconds.

To confirm this finding more clearly experimentally, reflectance was measured. Specifically, the reflected light intensity of dyed hair according to the coating times indicated in FIGS. 2 and 3 was measured using an optical microscope setup. The measurement of the reflected light intensity was performed using an Olympus BX53 optical microscope and an MPlanFL N 20× (numerical aperture 0.45) objective lens, and the reflected light was collected through an optical spectrometer (Ocean Optics QEpro or an equivalent spectrometer) mounted on the microscope.

A white light halogen lamp (PHILIPS 7724) was used as the light source, and hair samples were fixed on glass slides for measurement. The measured reflected light of the reference mirror (silver mirror) was set as the reference value, and the relative reflectance was calculated according to Equation 1 below:

$$\% {R}_{λ} = \frac{{S}_{λ} - {D}_{λ}}{{R}_{λ} - {D}_{λ}} \times 100 \% \qquad [ Equation 1 ]$$

wherein S: the reflected light intensity of the sample, D: the background signal, and R: the reflected light intensity of the reference reflector. The measurement wavelength range was set to 400 nm to 800 nm. Each spectrum was measured 10 times on average in different wavelength regions, and the measurements were averaged.

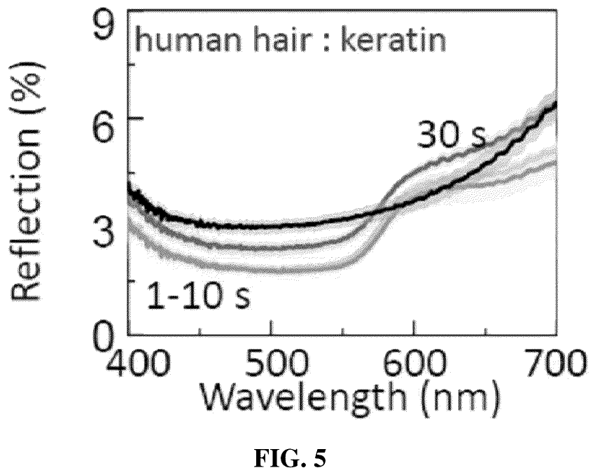

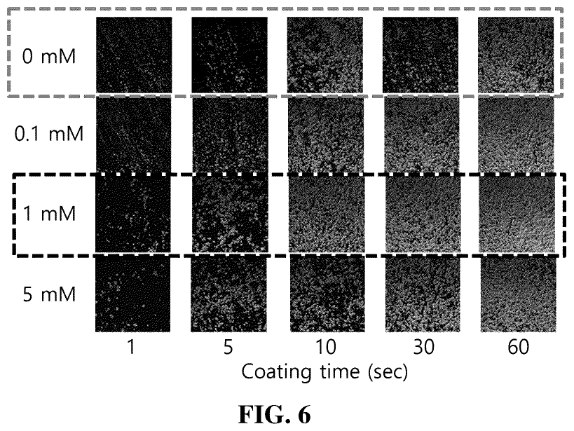

As a result of measuring the reflectance, it was confirmed that the hair exhibited a reflectance of up to 38% (700-nm wavelength) within 10 seconds (FIGS. 4 and 5).

### Evaluation of Hair Dyeing According to Concentration of Acidity Regulator

Hair dyeing was performed using dyeing solutions containing the acidity regulator sodium bisulfite at concentrations of 0 mM, 0.1 mM, 1 mM, and 5 mM while changing the dyeing time from 1 second to 5 seconds, 10 seconds, 30 seconds, and 60 seconds. Thereafter, changes in surface potential were evaluated and adherence rate was evaluated.

The dyeing solutions required for the dyeing process were prepared in the same manner as in the Preparation Example, except that the concentration of the acidity regulator was changed as described above.

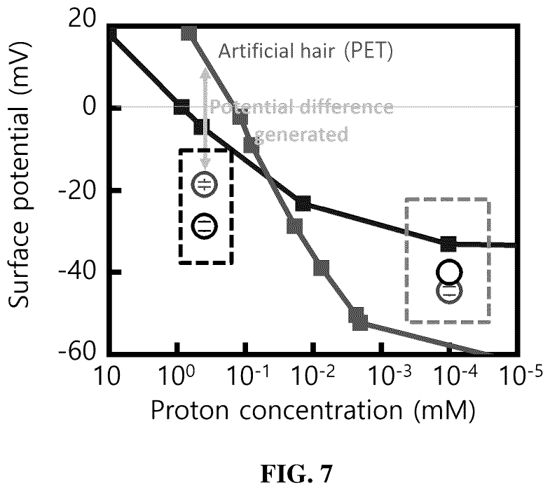

The experimental results are shown in FIGS. 6 to 9.

Referring to FIG. 6, it was confirmed that excellent hair dyeing effects were observed when 0.1 mM or more of the acidity regulator was contained, except when the acidity regulator was not contained. It was confirmed that the most preferable hair dyeing effect was observed when 1 mM of the acidity regulator was contained, but excellent hair dyeing effects were observed even at other concentrations of the acidity regulator.

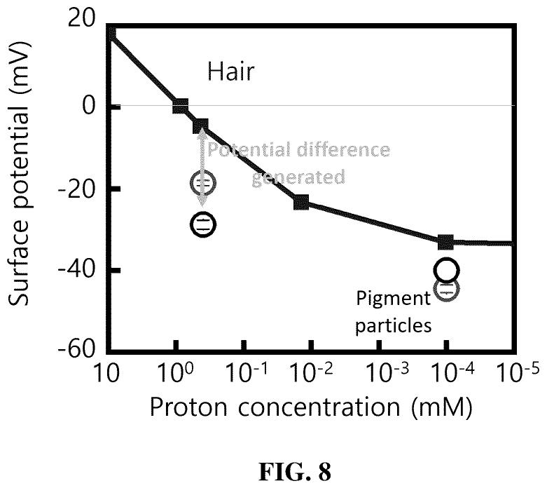

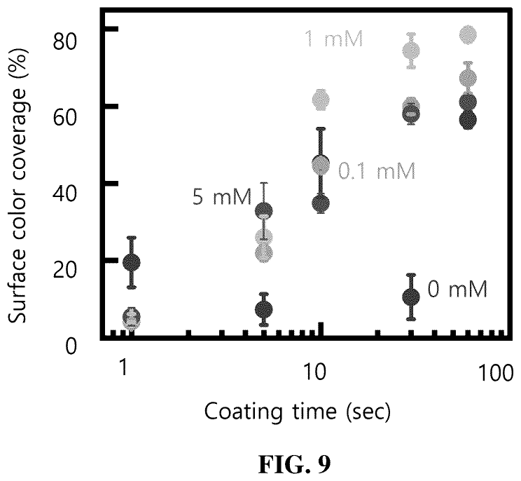

FIGS. 7 and 8 show the results of measuring the surface potential through zeta potential measurement of particles dispersed in the solution. Specifically, the measurement was performed using the product Zetasizer Nano ZSP (Malvern Instruments) while changing the proton concentration in the solution. A total of 10 analyses were performed for about 30 minutes, and the average value and error are shown.

Referring to FIGS. 7 and 8, the potential difference between the pigment particles and the hair can be confirmed. By the electrostatic attraction due to this potential difference, the pigment particles can be adsorbed on the hair and exhibit a hair dyeing effect.

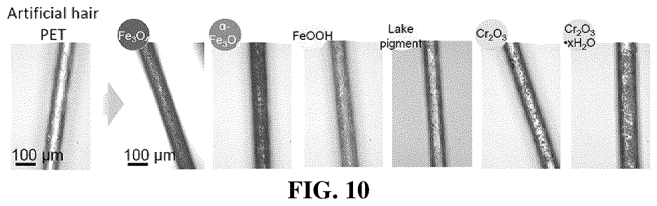

FIG. 9 shows images visually comparing the coverage of the coating layer formed on the hair surface as a function of coating time, and shows the results of analyzing the area based on the image. Optical bright-field and dark-field (BF/DF) images were acquired using a CCD camera (STC-MCS500U3V), and the samples were photographed under the same lighting conditions. The images are optical micrographs acquired under the same lighting conditions, and the coated area was isolated by extracting a red pixel value from each image. Image analysis was performed using ImageJ software (or an equivalent commercial/open-source image analysis program). The coated area was masked by setting the intensity threshold of the red channel, and then the ratio of the red area to the total area was calculated. The calculated ratio is defined as surface color coverage (%) according to the following equation:

$$Surface color coverage ( \% ) = \frac{{A}_{red}}{{A}_{total}} \times 100 \%$$

wherein A_{red} is the area of the region classified as a red pixel, and Aₜₒₜₐₗ is the total area to be analyzed in the entire image. The measurements were repeated three or more times in the same manner in different surface areas, and the average of the obtained values was defined as the adherence rate.

Referring to FIG. 9, it was confirmed that, when sodium bisulfite was contained at concentrations of 0.1 mM, 1 mM, and 5 mM, an excellent hair dyeing effect was achieved within a short period of time. In particular, it can be confirmed that, when sodium bisulfite was used at a concentration of 5 mM, hair dyeing was completed within 10 seconds.

### Evaluation of Color Change According to Difference in Pigment Particles

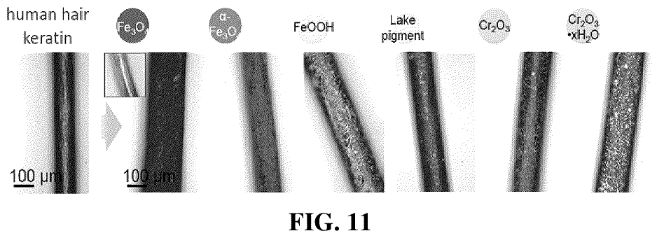

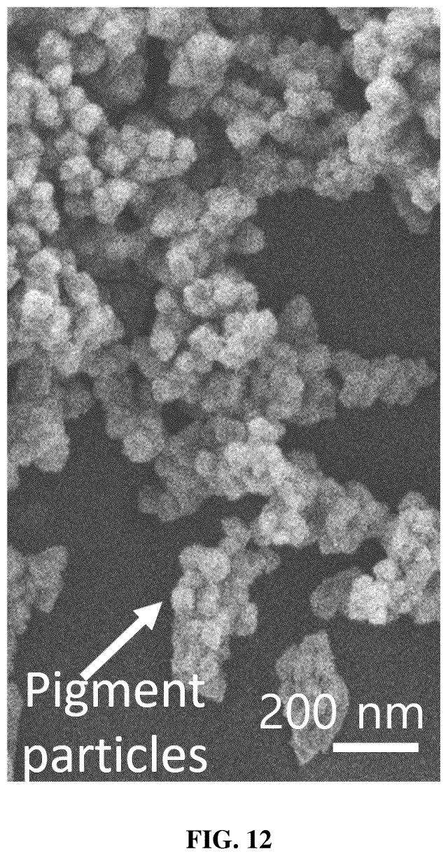

The color of hair dyed using dyeing solutions prepared by varying only the pigment particles in the Preparation Example was evaluated. The hair dyeing process was performed using artificial hair and actual human hair, and the results are shown in FIGS. 10 and 11.

FIG. 10 shows the dyeing results for artificial hair, and FIG. 11 shows the dyeing results for human hair. It was confirmed that black, red, yellow, and blue colors could be realized depending on the type of pigment particles, and that the colors were realized in the same way without any color difference depending on the difference in hair.

Hair Dyeing with Added Particle Fixing Layer

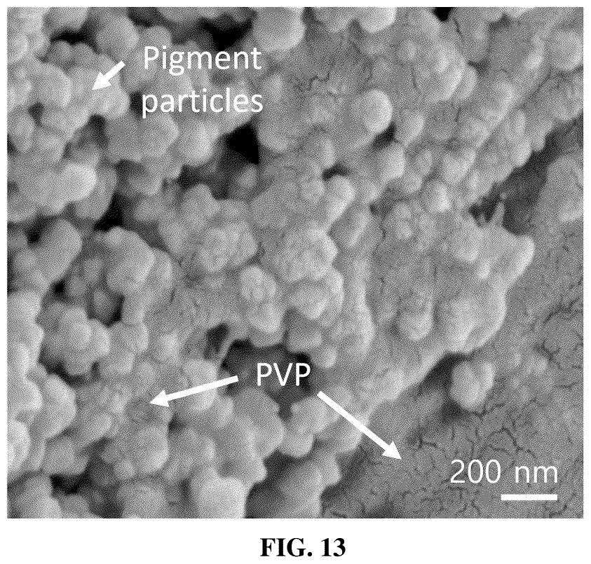

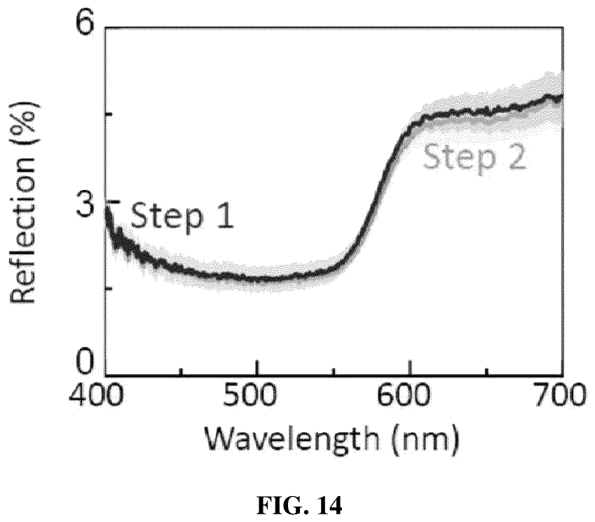

A hair dyeing process was performed using the dyeing solution prepared according to the Preparation Example, and a particle fixing layer was formed using a pigment particle fixing solution. Scanning electron microscope (SEM) images of hair before and after forming the particle fixing layer were checked. In FIG. 12, only the pigment particles were found because only the hair dyeing process was performed, whereas, in FIG. 13, it can be seen that the particle fixing layer was formed together with the pigment particles.

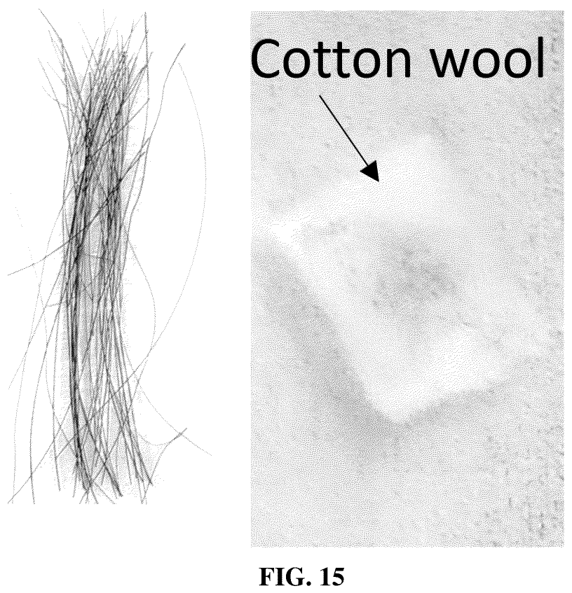

As a result of additionally checking whether there was a change in optical performance as the particle fixing layer was additionally formed, using the relative reflectance according to Equation 1 above (FIG. 14), it was confirmed that the optical performance was equivalent in both the hair on which only the pigment particles were adsorbed and the hair including the pigment particles and the particle fixation layer adsorbed thereon.

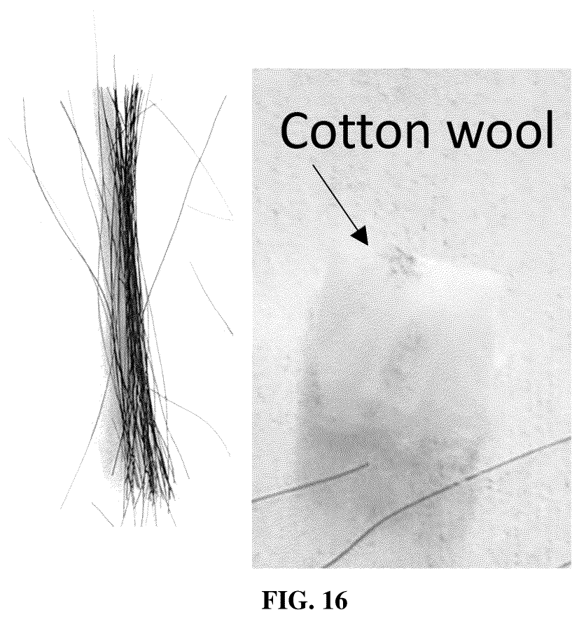

In addition, in order to check whether the pigment particles fall off due to external impact, the dyed hair was rubbed with cotton wool and whether the pigment particles adhered to the cotton wool was checked. As a result, it was confirmed that, in the case of the hair to which only the pigment particles were adsorbed, many pigment particles adhered to the cotton wool (FIG. 15), whereas in the case of the hair including both the pigment particles and the particle fixing layer adsorbed thereon, there were almost no pigment particles on the cotton wool, indicating that durability was improved (FIG. 16).

Although the preferred embodiments of the present disclosure have been described in detail above, the scope of the present disclosure is not limited thereto, and various modifications and improvements made by those skilled in the art using the basic concept of the present disclosure as defined in the appended claims also fall within the scope of the present disclosure.

What is claimed is:

1. A method of dyeing hair, comprising:
a step of dyeing hair by immersing the hair in a dyeing solution containing an acidity regulator and pigment particles, causing the pigment particles to be physically adsorbed on the hair; and
a step of immersing the dyed hair in a pigment particle fixing solution,
wherein the step of dyeing the hair changes a surface potential of the hair by the dyeing solution, causing the pigment particles to be physically adsorbed on the hair.

2. The method of claim 1, wherein the step of immersing the dyed hair in the pigment particle fixing solution forms a particle fixing layer between the pigment particles, adsorbed on the hair, by the pigment particle fixing solution.

3. The method of claim 1, wherein the dyeing solution further contains citrate ions.

4. The method of claim 3, wherein the dyeing solution contains 0.01 mM to 20 mM of the acidity regulator, 0.01 mM to 100 mM of the citrate ions, and 0.1 wt % to 0.2 wt % of the pigment particles.

5. The method of claim 1, wherein the pigment particles are selected from the group consisting of Fe₃O₄, α-Fe₂O₃, FeOOH, Cr₂O₃, Cr₂O₃·xH₂O, lake pigment, and mixtures thereof.

6. The method of claim 1, wherein the pigment particles have a diameter of 500 μm or less.

7. The method of claim 1, wherein the acidity regulator is selected from the group consisting of sodium bisulfite, succinic acid, aminomethyl propanediol, acetic acid, disodium phosphate, triethanolamine, and mixtures thereof.

8. The method of claim 1, wherein the pigment particle fixing solution is selected from the group consisting of polyvinylpyrrolidone (PVP), polyvinyl alcohol (PVA), a cellulose derivative, chitosan, alginate, and mixtures thereof.

9. The method of claim 1, wherein a concentration of the pigment particle fixing solution is 20 wt % or less.

10. The method of claim 1, further comprising, after the step of dyeing the hair, but before the step of immersing the dyed hair in the pigment particle fixing solution, a step of washing the dyed hair.

11. The method of claim 1, further comprising, after the step of immersing the dyed hair in the pigment particle fixing solution, a step of washing the hair.

12. The method of claim 1, wherein the step of dyeing the hair by immersing hair in the dyeing solution containing the acidity regulator and the pigment particles, causing the pigment particles to be physically adsorbed on the hair, dyes the hair by immersing the hair in the dyeing solution for 60 seconds or less.

13. A hair dyeing composition comprising:
a dyeing solution containing an acidity regulator and pigment particles; and
a pigment particle fixing solution,
wherein a surface potential of hair is changed by the dyeing solution, causing the pigment particles to be physically adsorbed on the hair, so that the hair is dyed, and
color durability of the dyed hair is increased by immersing the dyed hair in the pigment particle fixing solution.

14. The hair dyeing composition of claim 13, wherein the dyeing solution further contains citrate ions.
